(19) United States
(12) Patent Application Publication
(10) Pub. No.: US 2010/0256675 A1
(43) Pub. Date: Oct. 7, 2010

(54) Absorbable surgical staple

(76) Inventor: Matthew L. Romans, Salinas, CA (US)

Correspondence Address: BRADLEY P. HEISLER;HEISLER & ASSOCIATES, 3017 DOUGLAS BOULEVARD, SUTIE 300, ROSEVILLE, CA, 95661, US

(21) Appl. No.: 12/798,500

(22) Filed: Apr. 5, 2010

Related U.S. Application Data
(60) Provisional application No. 61/211,816, filed on Apr. 3, 2009.

(51) Int. Cl. A61B 17/064

(52) U.S. Cl. 606/219

(57) ABSTRACT

A surgical staple is provided which has at least a portion thereof which is bio-absorbable. Portions of the staple are configured to retain the staple at a subcutaneous location. After absorption of portions of the staple, other portions of the staple can be removed. In one form, the staple includes an elongate cross bar with a pair of posts extending from ends of the cross bar to tips. One form of subcutaneous retainer is provided on the posts in the form of barbs oriented to hold the posts subcutaneously.

### CROSS-REFERENCE TO RELATED APPLICATIONS

This application claims benefit under Title 35, United States Code §119(e) of U.S. Provisional Application No. 61/211,816 filed on Apr. 3, 2009.

### FIELD OF THE INVENTION

The following invention relates to bio-absorbable tissue closing and grafting devices and methods for their attachment. More particularly, this invention relates to surgical tissue staplers for closing skin incisions, lacerations and securing skin grafts to flat surface wounds.

### BACKGROUND OF THE INVENTION

Surgical skin staples have been used for decades for rapid closure of surgical wounds or incisions. They are popular because of the rapidity with which they can be placed. Generally, they are made of stainless steel and when they are placed, the stapling device bends the staple to hook the tissue. A special staple remover is later used to “unbend” the staple allowing it to be removed later when the incision is healed. These staples have also been widely used to attach skin grafts to the surface of large wounds.

The disadvantage of surgical skin staples is that they have to be removed later. The holes that metal skin staples make through the epidermis can lead to permanent scarring, especially if left in too long. This staple removal procedure can take quite a lot of time for a health-care provider and can be painful for the patient. This is especially true for skin grafts done in extremely sensitive areas of the body such as the genitalia, or on very large areas of the body, such as in cases involving very larger burns. Skin grafts are often done in large or sensitive areas of the body and suturing on the skin graft can be very time consuming. Metal staples are very fast to use, but can be extremely tedious and painful to remove later. Thus, a need exists to improve on the methods and devices available in these cases.

Dissolvable skin staples have generally not been developed because of the lack of a dissolvable material that could be conformed or bent to hold the tissues together. One example of a non-bending surgical fastener is described in U.S. Pat. No. 5,618,311, issued to Gryskiewicz on Apr. 8, 1997 that uses an absorbable non-bending staple that is in the subcuticular space only. This device relies on the spring-like physical property of the material forming the C-shaped clip and has no bending interlocking ends to hold the staple in place. This device is useful for closing sub-surface portions of surgical incisions with a dissolvable staple that is buried under the skin and leaves no scars, but it is not usable to secure skin grafts or to secure superficial portions of incisions or wounds.

### SUMMARY OF THE INVENTION

This invention is a biodegradable staple that in a preferred form has two preferably rigid substantially vertical posts with sharp tissue-penetrating tips connected by a preferably rigid or semi-rigid horizontal component. The vertical posts are driven directly into the tissue and held in place by multiple barbs much like the barbs on a fishhook (FIG. 1). The number and size of the barbs might vary between a few larger barbs and numerous smaller barbs, whatever strikes the best balance between allowing the staple to be driven into the tissue easiest while still holding onto the tissue. One form of such barbs are described in conjunction with QUILL SRS suture manufactured by Angiotech Pharmaceuticals, Inc. of Vancouver, British Columbia, Canada. There is no need for any deformity or bending of the staple to hold it in place. There is no need for the tips of the vertical posts to bend and interlock with each other.

The staple could be applied to the tissue with a device that is similar to that which drives staples into wood (e.g. to staple tar paper to a wall). The process would involve stabilizing the tissue with tissue forceps while an assistant uses the device to drive the staple into the tissues across the incision with one vertical post on either side (FIG. 2).

Because the barbs or hooks would hold the vertical posts in the tissue, there is no need for the staple to bend in order to grab the tissue. The physical characteristics of the staple will typically be rigid enough to drive into the tissue, especially if the staple is used to close incisions and penetrate epidermis and dermis in the same way that metal staples are used now. The staple would not have to be as rigid to drive it through a skin graft into subcutaneous tissue or muscle in order to hold a skin graft in place. The skin graft could be staples in with the staples around the edge are positioned parallel to the edge of the wound and with some staples in the middle of the wound.

Ideally, the staple would be made either of a substance that biodegrades and allows the horizontal component to detach and fall off within one to two weeks, or is caused to fall of by the application of a non-toxic substance that causes the horizontal device to dissolve and fall off. One example of such a substance might be polydioxanone. If the absorption time of this would be too long, a better material might be poliglecaprone 25 (a glycolide and e-caprolactone copolymer) because the tissue absorption times would be much shorter. Whatever the physical substance used, a typical absorption time would be somewhere in the neighborhood of one to three weeks.

One of the drawbacks of staples through the skin is that the longer the staples are left in, the more scarring the staple holes themselves cause. One solution to this would be to have zones of weakness just below the skin on the vertical posts that dissolve more quickly or is slightly weaker that allows the horizontal limb to fall off and detach just below the skin line, allowing more rapid re-epithelialization of the skin over the remaining vertical posts, and minimizing scarring.

The dimensions of the staple would be variable, but generally similar in size to metal staples. Currently, most metal staple manufacturers make skin staples in both “regular” and “wide” sizes, with the wide staples being the most commonly used.

### OBJECTS OF THE INVENTION

Accordingly, a primary object of the present invention is to provide a surgical staple which can be used to hold skin together, such as adjacent an incision or a graft until the healing process can naturally hold the skin in position.

Another object of the present invention is to provide a surgical staple which does not require bending of posts of the staple relative to a cross bar of the staple, during or after implantation, to hold the staple in place.

Another object of the present invention is to provide a surgical staple which minimizes scarring.

Another object of the present invention is to provide a surgical staple which includes barbs on posts thereof to retain the posts subcutaneously and keep the staple in a desired position.

Another object of the present invention is to provide a surgical staple with at least portions thereof which are bio-absorbable, such that they do not require removal, but rather can be absorbed into the body of the patient over time.

Another object of the present invention is to provide a method for closing an incision and holding skin grafts in place which is effective, easy to use, can be used in a minimal amount of time and avoids the necessity of return visits for removal of anything later.

Other further objects of the present invention will become apparent from a careful reading of the included drawing figures, the claims and detailed description of the invention.

### BRIEF DESCRIPTION OF THE DRAWINGS

FIG. 1 is a perspective view of a surgical staple including barbs thereon and optionally having at least portions thereof formed of absorbable materials, according to a first embodiment of this invention.

FIG. 2 is a front elevation view of that which is shown in FIG. 1.

FIG. 3 is a sectional view taken along lines 3-3 of FIG. 2 and illustrating further details of barbs provided on the surgical staple of this embodiment.

FIG. 4 is a front elevation view of the surgical staple of FIG. 1 shown implanted within a patient in a position closing an incision in the patient's skin.

FIG. 5 is a top plan view of an entire incision on a torso of a patient and illustrating how multiple surgical staples according to one embodiment of this invention can be utilized to close such an incision.

FIG. 6 is a top plan view of a skin graft site illustrating a method for utilizing the surgical staple of one embodiment of this invention to secure a margin and central portion of the skin graft in position.

FIG. 7 is a perspective view of an alternative embodiment surgical staple featuring a zone of weakness according to an alternative embodiment of this invention.

FIG. 8 is a front elevation view of that which is shown in FIG. 7, with the alternative surgical staple shown in an implantation site and after the zone of weakness has been allowed to separate so that the cross bar can be removed form posts of the surgical staple, and illustrating how the epidermis can effectively close over the area where the zone of weakness was previously located to minimize scarring.

FIG. 9 is a front elevation view of a further alternative embodiment of the surgical staple of this invention illustrating an alternative barb pattern as well as exemplary dimensions for one embodiment surgical staple according to this invention.

### DESCRIPTION OF THE PREFERRED EMBODIMENT

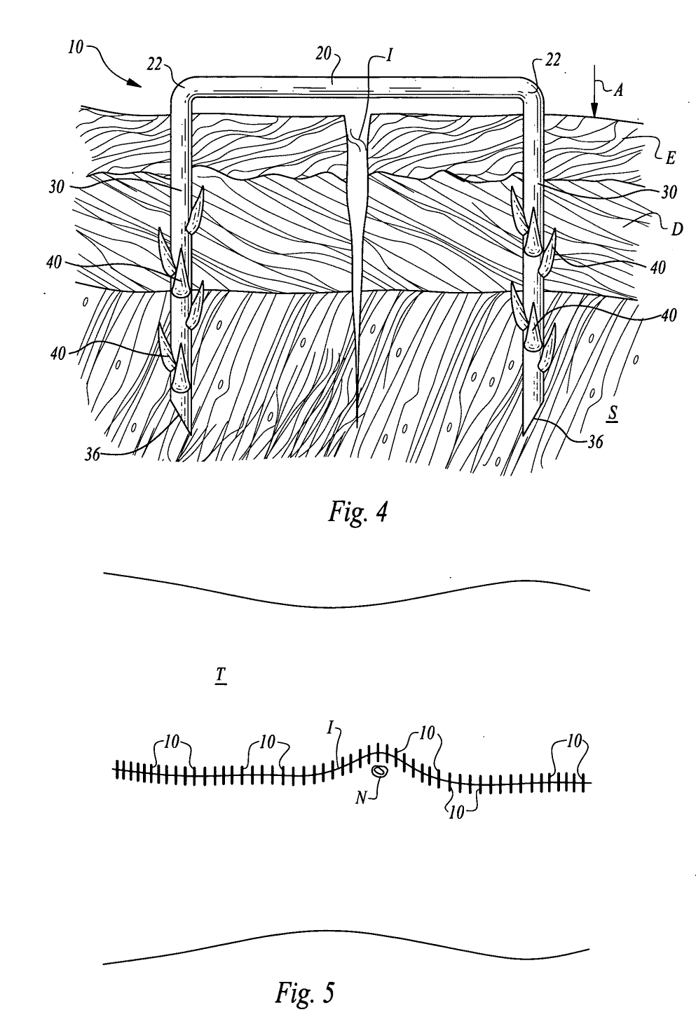

Referring to the drawings, wherein like reference numerals represent like parts throughout the various drawing figures, reference numeral 10 is directed to a surgical staple as a preferred form of suturing device for use in holding closed incisions I (FIG. 4) in a simple and highly effective manner. In a preferred embodiment, the staple 10 includes barbs 40 which keep the staple 10 positioned within the skin of the patient, and resisting inadvertent removal thereof. At least portions of the staple 10 are preferably absorbable, such as by forming portions of the staple from a bio-absorbable material, to minimize or eliminate the necessity to later remove portions of the staple 10 and to minimize scarring.

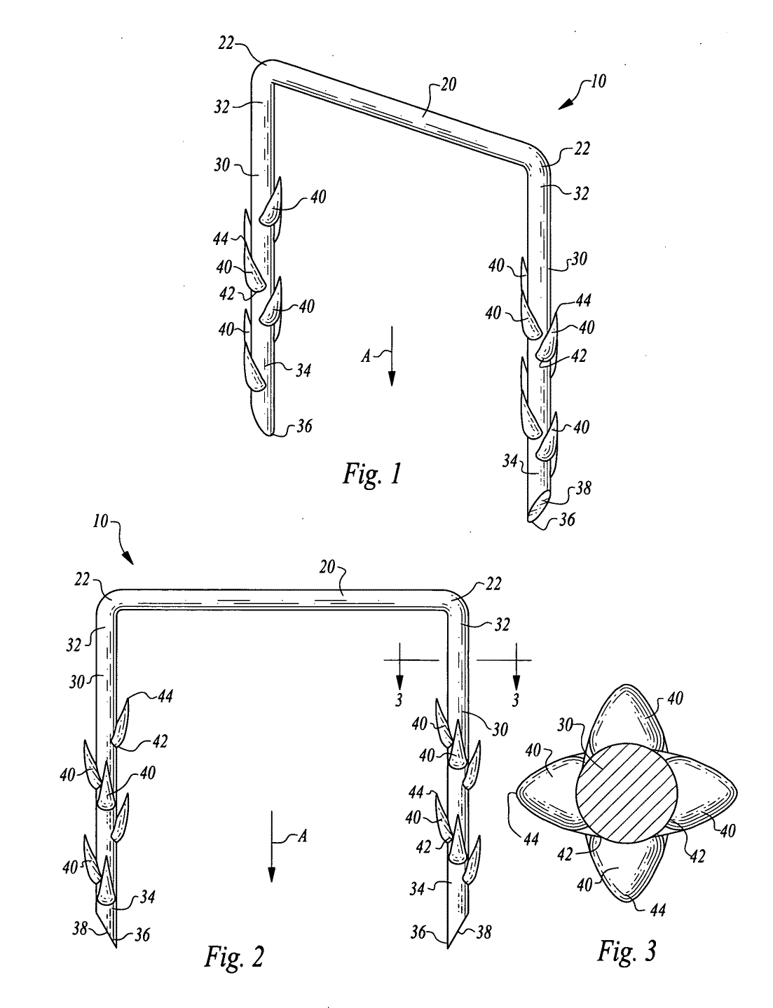

In essence, and with particular reference to FIG. 1, basic details of the surgical staple 10 of a preferred embodiment of this invention are described. The staple 10 of this embodiment includes an elongate cross bar 20 defining a portion of the staple 10 which typically remains outside of the patient's skin. A pair of posts 30 extend from the cross bar 20, preferably substantially perpendicular to the cross bar 20 and substantially parallel to each other. These posts 30 include barbs 40 as a preferred form of subcutaneous retainer on the posts 30. The barbs 40 are configured to allow the posts 30 to pass easily into the skin of the patient (along arrow A of FIG. 1) while resisting removal of the staple 10 and associated posts 30, should forces be applied to the staple 10 tending to remove the staple 10 out of the skin of the patient.

More specifically, and with continuing reference to FIG. 1, details of the cross bar 20 are further described according to this preferred embodiment and alternatives thereof. The cross bar 20 is preferably a substantially rigid linear elongate structure having a substantially constant cross-sectional form between ends thereof. These ends support bends 22 preferably formed from the same material forming the cross bar 20, with the bends 22 bending approximately 90° and transitioning into upper ends 32 of the posts 30.

While the cross bar 20 preferably has this configuration shown in FIG. 1, various alternatives could be provided for the cross bar 20 within the scope of this invention. For instance, the cross bar 20 could have an irregular cross section, such as with flattened portions facing upwardly, or contours which are particularly configured to engage with an application tool. For instance, the cross bar 20 could have a contour which closely matches a contour in an application tool so that the cross bar would snap into a groove or other recess in the application tool and the staple would be held to the application tool. The staple 10 could then be implanted where desired and the application tool then allowed to separate from the cross bar 20.

While the cross bar 20 is preferably rigid, such as being formed of a stainless steel, preferably of a bio-compatible variety, the cross bar 20 could conceivably be formed of a material which exhibits varying degrees of flexibility and potentially even elasticity. For instance, the cross bar 20 could function according to this invention in the form of a flexible line securing the two posts 30 together. The posts 30 might conceivably be implanted separately on opposite sides of an incision I (FIG. 4) with the cross bar 20 merely joining the upper ends 32 of the posts 30 together to provide a compression force tending to hold the upper ends 32 of the posts 30 together and tending to draw the incision I or other margins closed together.

The cross bar 20 could exhibit both flexibility and potentially also elasticity, such that the cross bar 20 could be stretched somewhat before implantation and then forces within the material itself tending to return the cross bar 20 to its original length would continue to maintain a closing force across the incision I. In such a flexible embodiment, the cross bar 20 could be formed of materials typically utilized in forming sutures or from other materials having suitable flexibility and elasticity properties desired by the user.

Most preferably, the bends 22 are at the extreme ends of the cross bar 20. However, the cross bar 20 could conceivably extend beyond these bends 22. Most preferably, these bends 22 orient the posts 30 perpendicular to the cross bar 20. However, these bends 22 or other junctions between the posts 30 and the cross bar 20 could have other angular dimensions so that the posts 30 would have other orientations relative to the cross bar 20 other than perpendicular. At a minimum, the posts 30 are oriented non-parallel with the cross bar 20 and substantially in a common plane with the cross bar 20.

Preferred dimensions for the cross bar 20 include a length of between five and seven millimeters in a most preferred form. However, the cross bar 20 could have a greater or lesser length than this most preferred range to suit the particular needs of a user. Preferably, the cross bar 20 has a width substantially ten percent of a length of the cross bar 20. However, this ratio of thickness to length of the cross bar 20 could be modified outside of this preferred range and still function effectively according to this invention. Thus, it can be seen that while the figures illustrate a simple and straightforward preferred form for the cross bar 20, the cross bar 20 could be altered as described herein and still function according to this invention.

With continuing reference to FIG. 1, as well as FIGS. 2-4, specific details of the posts 30 of this invention are described, according to this most preferred embodiment. Each of the posts 30 are preferably of similar form to each other and exhibit an elongate linear substantially constant cross-sectional form extending between the upper end 32 and a lower end 34 opposite the upper end 32. The upper end 32 is preferably located adjacent the bends 22 in the cross bar 20. In a most preferred embodiment, this transition between the upper end 32 of each post 30 and the bends 22 of the cross bar 20 is substantially continuous and not defined by any particular transition point.

Each post 30 includes a tip 36 at its lower end 34. This tip 36 causes the post 30 to have a smaller width at the tip 36 than other portions of the post 30. One technique for forming this tip 36 is to provide a facet 38 adjacent the lower end 34 at an angle skewed relative to a central axis of the posts 30. This facet 38 is preferably configured so that the tips 36 are on an inner side of each post 30. However, the facet 38 could have other orientations so that the tip 36 would be on an outer side of each post 30, or front, rear or other orientation.

As another alternative, the tip 36 could be sharpened to a point at or near the post 30 central axis. By providing the tip 36 sharpening to a small width, the post 36 is configured to most easily pass into the skin of the patient transcutaneously, with a minimum of distress caused to the patient. By providing the tips 36 on inner sides of the posts 30, the posts 30 are tended to be drawn slightly toward each other, thus to a small extent exerting a force drawing the posts 30 toward each other (FIG. 4), in one typical use of the staple 10 of this invention.

The posts 30 are preferably rigid and have a substantially constant cross-sectional form. However, the posts 30 could conceivably be to at least some extent flexible and could have a small degree of variability in cross-section. The cross-section could be round as depicted herein or have some other contour, such as a square, rectangle or polygonal contour, or some form of oval contour.

Most preferably, the two posts 30 extend parallel to each other a common distance away from the cross bar 20. However, the posts 30 could have lengths which differ from each other to suit particular applications where posts of differing lengths might be considered suitable. For instance, subcutaneous anatomy (e.g. a bone) that is to be avoided might indicate use of a staple 10 to both maximize post 20 depth while still avoiding the anatomical structure(s) the medical professional desires to avoid. The posts 30 preferably reside within a common plane with each other and also with the cross bar 20.

The posts could be formed of a variety of different bio-compatible materials, with bio-compatibility particularly important for the posts 30 due to their intended implantation subcutaneously. Most preferably, the material forming the posts 30 is a bio-absorbable material which is both bio-compatible and bio-absorbable. By “bio-compatibility,” it is intended that the material not cause the body to consider the posts 30 to be a foreign object which would lead to infection or other undesirable bodily response tending to reject the presence of the posts 30 subcutaneously. By “bio-absorbable,” it is intended to define materials which do not require removal from the body, but rather which over time disintegrate subcutaneously. Such disintegration could be in the form of a dissolving type process or some form of conversion process where the material forming the posts 30 is transformed into some other material or remains in position but in a manner which is of no long term negative effect on the patient by remaining therein. To qualify as absorbable, at least some amount of this absorbability would occur, but not necessarily complete absorbtion.

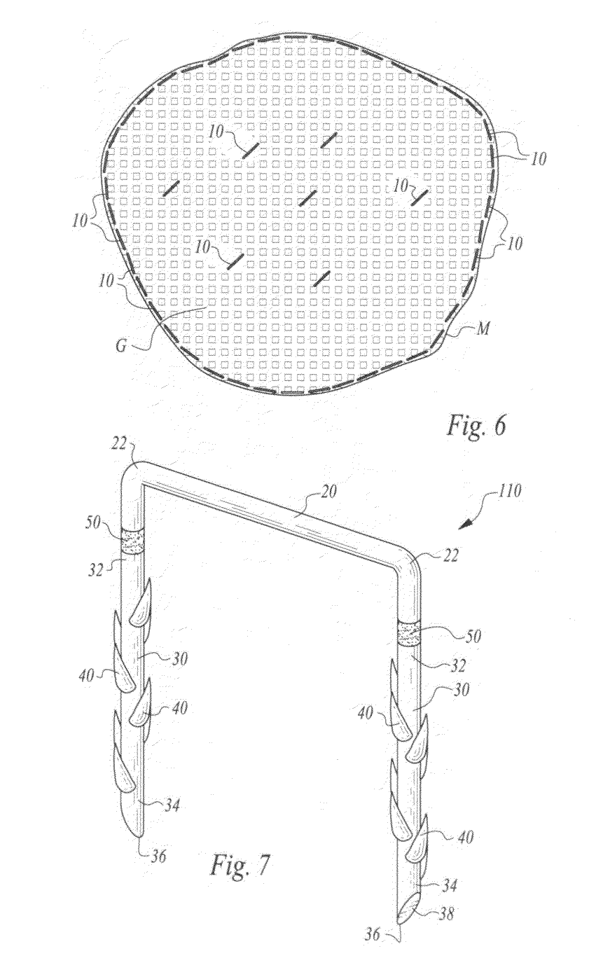

It is conceivable that the posts 30 could be formed from different materials with some materials being bio-absorbable and other portions of the posts 30 not being bio-absorbable. For instance, portions of the posts 30 adjacent the upper end 32 might be formed of a bio-compatible but not bio-absorbable material, intended to be removed along with the cross bar 20 after the incision I or graft G (FIG. 6) has healed sufficiently. Such embodiments are described in further detail below. At the same time, portions of the posts 30 closer to the tip 36 could be formed of a material which is both bio-compatible and bio-absorbable.

With continuing reference to FIG. 1 as well as FIGS. 2-4, particular details of the barbs 40 defining a preferred form of subcutaneous retainer for the posts 30, are described according to this preferred embodiment. The barbs 40 are provided with at least one barb on at least one post 30. Preferably, each post 30 includes at least one barb 40, and most preferably each post 40 includes a plurality of such barbs 40 on various different sides of each of the posts 30.

In this preferred embodiment, each barb 40 includes a root 42 spaced from a point 44. The root 42 defines a portion of each barb 40 which is connected to the post 30, with the point 44 defining that portion of each barb 40 most distant from the root 42. The point 44 of each barb 40 is located closer to the cross bar 20 than the root 42 of each barb 40. Thus, when removal forces (opposite arrow A of FIGS. 1 and 4) are applied to the posts 30, the points 44 of the barbs 40 dig into surrounding subcutaneous tissue S or dermal tissue D and resist such removal motion. However, the barbs 40 provide minimal resistance to insertion forces (along arrow of FIGS. 1 and 4) involved in the implantation of the staple 10.

Each of the barbs 40 is most preferably formed by making a cut into the post 40 at an angle (e.g. 25°) skewed relative to the central axis of the posts 30. This cut is only made partially into the post 30. This cut is associated with a bending action so that a portion of the post 30 on one side of each cut is bent away from the central axis of the post, leaving the barb with the point 44 pointing toward the cross bar 20 at least somewhat.

Preferably, the barbs 40 are spaced from each other, such as in a spiral pattern extending helically around the posts 30 along a path extending from near the upper end 32 to near the lower end 34, so that barbs 40 are uniformly spaced on various different sides of the posts 30. As an alternative (FIG. 9) the barbs 40 can be provided in opposing configuration with barbs 40 provided in balanced pairs on opposite sides of each post 30 of an alternative staple 210, and preferably with the orientation of these opposing pairs of barbs 40 alternating between spaced within a plane including the cross bar 20 and posts 30, and pairs of barbs 40 in a common plane perpendicular to the plane including the posts 30 and cross bar 20.

In this embodiment, each barb 40 has a length from the root 42 to the point 44 which is approximately one-fifth of a length of each post 30. However, the barbs 40 could be made smaller or larger than this exemplary size.

The barbs 40 provide a preferred form of the subcutaneous retainer for the posts 30. Other means to hold the posts 30 subcutaneously after implantation of the staple 10 could also conceivably be utilized. For instance, bumps on sides of the posts 30 extending lateral to a long axis of the posts 30 that would resist removal of the posts 30 (in a direction opposite arrow A of FIG. 1) and could be provided as an alternative to the barbs 40. While such bumps would exhibit a similar degree of resistance to passage both into the skin (along arrow A of FIG. 1) as out of the skin (in a direction opposite arrow A of FIG. 1) after the skin and subcutaneous tissues S have received the post 30 with such bumps therein, an enhanced amount of force would be required to remove the staple 10 over what would be required with a staple 10 with posts 30 having no bumps or barbs 40 thereon.

Most preferably, the barbs 40 or other subcutaneous retainers on the posts 30 are formed from a common material with the posts 30 which material is a bio-compatible and bio-absorbable material. As an alternative, the barbs 40 or other subcutaneous retainers could be formed of a material which is bio-compatible and bio-absorbable, while the post 30 would be formed of a bio-compatible material which is not bio-absorbable. In such an embodiment, the barbs 40 or other subcutaneous retainers would continue to hold the staple 10 at the implantation site until the time associated with bio-absorbability has elapsed. The entire staple 10 including the posts 30, but not including the barbs 40 or other subcutaneous retainer could then be removed. Most preferably, however, the posts 30 are absorbable along with the barbs 40 or other subcutaneous retainers, so that the posts 30 need not be removed after the patient has healed.

With particular reference to FIG. 7, details of an alternative surgical staple 110 are described which features zones of weakness 50 on upper portions of the posts 30 adjacent the cross bar 20. With this alternative staple 110, the geometric configuration of the staple 10 can be similar to previous embodiments described herein or could be modified according to alternatives described elsewhere herein. Uniquely with this embodiment, a zone of weakness 50 is provided at the upper end 32 of each of the posts 30. This zone of weakness 50 can be weakened geometrically, such as by providing a lesser width at the zone of weakness 50. By making the zone of weakness 50 having a lesser width, it inherently exhibits lesser strength characteristics. If the post 30 is also formed of bio-absorbable material, this zone of weakness 50 having a lesser geometric form would absorb more quickly.

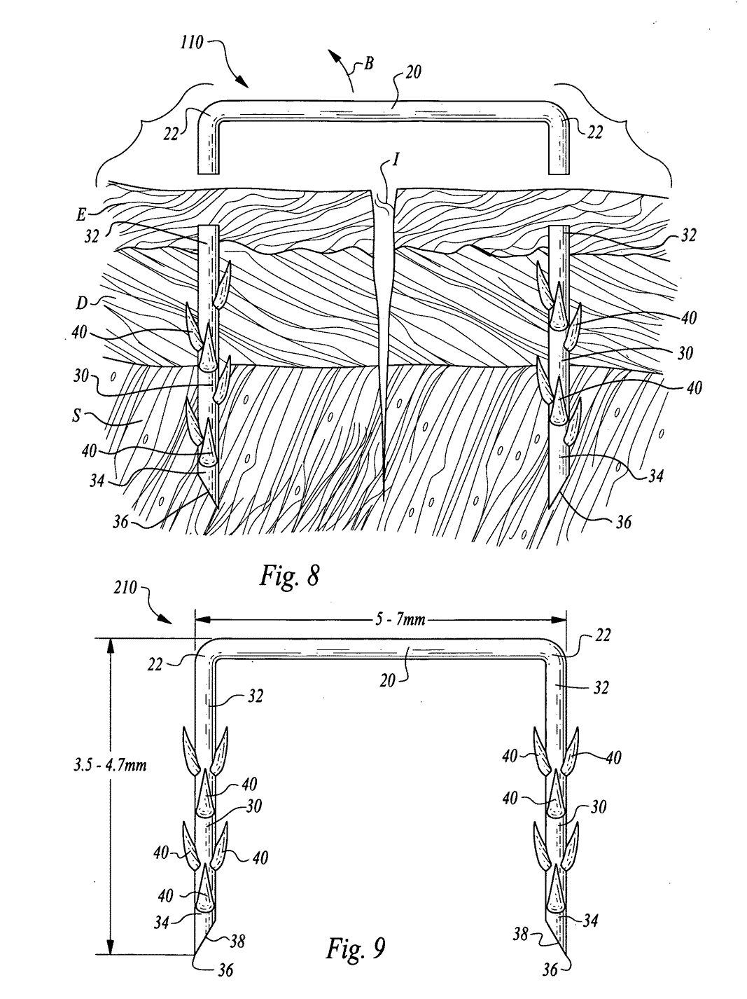

As another alternative, the zone of weakness 50 can be provided by utilizing an alternative material at the zone of weakness 50. For instance, a material which has a higher rate of bio-absorbability then other portions of the posts 30 could be utilized. In such an embodiment, even though the geometry of the zone of weakness 50 is sufficient and similar to geometry of adjacent portions of the posts 30, the higher rate of bio-absorbability causes this zone of weakness 50 to weaken more rapidly as the material forming the zone of weakness 50 absorbs. Such an embodiment facilitates functions such as that illustrated in FIG. 8, where the epidermis E can conceivably begin to seal over and through the location of the zone of weakness 50, as the zone of weakness 50 absorbs before other portions of the staple 110. The cross bar 20 can then be removed (along arrow B of FIG. 8). However, other portions of the posts 30 and barbs 40 remain in a subcutaneous position. Over time, these posts 30 and barbs 40 would also preferably bio-absorb, but the relatively rapid bio-absorbability with the zone of weakness 50 causes the epidermis E to rapidly heal at the site where the posts 30 penetrated the epidermis E, for minimal scarring.

As another alternative, the zone of weakness 50 could be formed of a material which is configured to react to a particular solvent. For instance, a material could be selected which dissolves when exposed to rubbing alcohol. When a medical professional has determined that the incision I has healed sufficiently that the staples 110 are no longer required, the medical professional, or through instructions to the patient or others, could apply rubbing alcohol to the skin adjacent the staple 10. The rubbing alcohol would react with the zone of weakness 50 and cause the zone of weakness to absorb, thus allowing the cross bar 20 portion of the surgical staple 110 to be readily removed and completing the incision I healing process, while allowing subcutaneous portions of the posts 30 and barbs 40 to bio-absorb over time later. Other dissolving agents could similarly be utilized with the material forming the zone of weakness 50 appropriately modified to react appropriately to the dissolving material selected.

With particular reference to FIGS. 4-6 and 8, details of the use of the surgical staple 10 of this invention to close an incision I or wound, or to hold a skin graft G in place, are described according to this preferred embodiment. Initially, an incision I or other site is identified which benefits from margins being closed together. An exemplary such incision is shown in elevation in FIG. 4 and in a top plan view in FIG. 5. Utilizing forceps, or the user's hands or the hands of an assistant, or other retraction equipment, the two margins of the incision I are brought together. Next, a staple 10 is placed with one of the posts 30 on a first side of the incision I and the other post 30 on the opposite side of the incision I.

The staple 30 could conceivably be applied merely be pressing the staple 10 into the skin. As an alternative, some form of staple tool could be utilized to drive the staple into the skin. Such a tool could be similar to tools utilized to staple paper to a wall. The staples could be provided in some form of array which holds a plurality of staples together and with a driver configured to only drive one of the staples in the array, in a sequential fashion, such that stapling can rapidly occur after the two sides of the incision have been brought together.

The spacing of multiple staples 10 can be according to the skill of a medical professional or according to some precalculated preferred spacing instructions. Once all the staples 10 have been placed, the incision I has been completely closed. Typically, a final cleaning procedure is provided and some form of dressing is provided over a top of the entire incision I. If required, drain tubes can be provided as is known in the art. While the incision I is shown in FIG. 5 on the torso T of a body, such as following a line adjacent a navel N of the torso T, the incision I could be on any portion of the body and benefit from the staples 10 of this invention and applied according to the method of this invention.

When a skin graft G is being applied to a space, the staples 10 of this invention can be utilized to hold the graft G in position. In particular, the skin graft G can be placed over an area where the skin graft G is to reside. The staples 10 are then placed about a margin M of the graft G to hold edges of the graft G in place. Especially for larger grafts G, staples 10 can also be utilized inboard of the margin M to hold the graft G to contours of the body of the patient. While the staples 10 are shown herein generally parallel with the margin M, it is conceivable that some of the staples 10 could be provided perpendicular to the margin M and crossing from the margin M to adjacent tissues if such a configuration is called for by the particular graft G implantation procedure.

When the surgical staple 110 is utilized having the zones of weakness 50, the same implantation procedure can be utilized, except that after the implantation procedure has been completed, and sufficient time has passed for the zone of weakness 50 to dissolve away (or be utilized as a weak point to break away the cross bar 20), the cross bar 20 can be removed and the epidermis E layer of the skin can grow over a top of the posts 30 to minimize scarring. After the staples 10, 110, 210 have been implanted, and sufficient time has been allowed to pass, non-bio-absorbable portions of the staples 10 are removed and bio-absorbable portions of the staple 10, 110, 210 are allowed to absorb in situ.

This disclosure is provided to reveal a preferred embodiment of the invention and a best mode for practicing the invention. Having described the invention in this way, it should be apparent that various modifications can be made to the preferred embodiment without departing from the scope and spirit of this invention disclosure. When structures are identified as a means to perform a function, the identification is intended to include all structures which can perform the function specified. When structures of this invention are identified as being coupled together, such language should be interpreted broadly to include the structures being coupled directly together or coupled together through intervening structures. Such coupling could be permanent or temporary and either in a rigid fashion or in a fashion which allows pivoting, sliding or other relative motion while still providing some form of attachment, unless specifically restricted.

What is claimed is:

1. A barbed staple, comprising in combination:
a cross bar having an elongate form between opposing ends thereof, said ends spaced apart by a length of said cross bar;
a first post coupled to said cross bar and extending away from said cross bar, said first post extending to a tip spaced from said cross bar by a length of said first post;
a second post coupled to said cross bar and extending away from said cross bar, said second post extending to a tip spaced from said cross bar by a length of said second post;
at least two barbs, with at least one said barb coupled to each of said posts, each said barb including a root spaced from a tip, with said root fixed to one of said posts and said tip spaced from said root by a length of said barb; and
said at least two barbs oriented with said tips of said barbs closer to said cross bar than said roots of said barbs.

2. The barbed staple of claim 1 wherein said first post and said second post are oriented substantially parallel to each other.

3. The barbed staple of claim 2 wherein said first post and said second post are each oriented substantially perpendicular to said cross bar.

4. The barbed staple of claim 1 wherein zones of weakness are interposed between said posts and said cross bar, such that said posts are coupled to said cross bar through said zones of weakness, said zones of weakness adapted to be weaker than portions of said posts and said cross bar adjacent said zones of weakness.

5. The barbed staple of claim 4 wherein said zones of weakness are adapted to allow said cross bar to be separated from said posts, with said posts adapted to remain within a patient while said cross bar is removed.

6. The barbed staple of claim 4 wherein said zones of weakness are formed of a material which is adapted to be absorbed within the body of a patient after said zones of weakness are implanted within the body of the patient as part of the staple.

7. The barbed staple of claim 6 wherein said zones of weakness are formed of a material which absorbs more rapidly into the body of the patient than a material forming other portions of the barbed staple.

8. The barbed staple of claim 1 wherein at least portions of said barbs coupled to said posts are adapted to absorb into the body of a patient after implantation within the body of the patient.

9. The barbed staple of claim 8 wherein said first post, said second post and said at least two barbs coupled to said posts are each formed of a common material adapted to be absorbed into the body of a patient after implantation therein.

10. An absorbable staple, comprising in combination:
a cross bar having an elongate form between opposing ends thereof, said ends spaced apart by a length of said cross bar;
a pair of posts including a first post and a second post, said posts coupled to said cross bar at locations on said cross bar spaced from each other, with said pair of posts each extending away from said cross bar to tips of said posts spaced from said cross bar;
at least one of said posts including a subcutaneous retainer thereon, said subcutaneous retainer adapted to hold the post in a subcutaneous position after implantation of the absorbable staple through skin of the patient; and
at least a portion of said subcutaneous retainer formed of an absorbable material adapted to be absorbed into the body of the patient after implantation therein.

11. The absorbable staple of claim 10 wherein both said posts and said subcutaneous retainer are each formed of a material that is adapted to be absorbed after implantation into the body.

12. The absorbable staple of claim 11 wherein each of said posts includes a subcutaneous retainer thereon, with both said posts and said subcutaneous retainers each formed of a common material adapted to be adsorbed into the body after implantation therein.

13. The absorbable staple of claim 10 wherein said subcutaneous retainer includes at least one barb, said barb including a root spaced from a tip, with said root affixed to said post and said tip spaced from said root by a length of said barb, said at least one barb oriented with said tip of said barb closer to said cross bar than said root of said barb.

14. The absorbable staple of claim 13 wherein both said barbs and said posts are formed of a common material adapted to be absorbed after implantation into the body of the patient.

15. The absorbable staple of claim 14 wherein zones of weakness are provided between each of said posts and said cross bar, said zones of weakness adapted to be weaker than portions of said cross bar and said posts adjacent said zones of weakness.

16. The absorbable staple of claim 15 wherein said zones of weakness are formed of a material adapted to be absorbed into the body after implantation therein, said zones of weakness adapted to absorb more rapidly than said posts adjacent said zone of weakness, such that said zones of weakness absorb first, and said cross bar can be removed while said posts continue to stabilize tissues through which said post is oriented, such as to minimize epidermal scarring.

17. A method for holding an incision closed, including the steps of:
identifying a barbed staple having a cross bar with an elongate form between opposing ends thereof, the ends spaced apart by a length of the cross bar; a first post coupled to the cross bar and extending away from the cross bar, the first post extending to a tip spaced from the cross bar by a length of the first post; a second post coupled to the cross bar and extending away from the cross bar, the second post extending to a tip spaced from the cross bar by a length of the second post; at least two barbs with at least one barb coupled to each of the posts, each barb including a root spaced from a tip, with the root coupled to one of the posts and the tip spaced from the root by a length of the barb; and the at least two barbs oriented with the tips of the barbs closer to the cross bar than the roots of the barbs; and
placing the barbed staple with the first post on a first side of the incision and the second post on a second side of the incision opposite the first side, with each of the posts penetrating into the skin on opposite sides of the incision and with the cross bar spanning the incision.

18. The method of claim 17 wherein said identifying step includes identifying the barbed staple having at least portions of the barbs formed of a material that is absorbable within the body after implantation therein; and
allowing at least portions of the barbs to be absorbed at a time sufficiently after said placing step that the incision has healed sufficiently to remain closed.

19. The method of claim 18 including the further step of removing at least the cross bar of the barbed staple after said allowing step.

20. The method of claim 19 wherein said removing step includes the step of removing both the cross bar and at least portions of the posts after said allowing step.

21. The method of claim 18 wherein said identifying step includes the step of identifying the barbed staple as one having zones of weakness between the cross bar and each of the posts, the zones of weakness formed of a material weaker than a material forming the cross bar and portions of the posts adjacent the zones of weakness; and
separating the cross bar above the zones of weakness from the posts below the zones of weakness after said placing step.

22. The method of claim 21 wherein said zones of weakness are formed of a material which is adapted to be absorbed into the body of the patient after implantation therein, with the zones of weakness formed of a material which absorbs more rapidly into the body than other portions of the barbed staple.

23. A skin suturing device, comprising in combination:
an elongate length of material extending between a first end and a second end;
a first subcutaneous retainer affixed to said length closer to said first end than said second end;
a second subcutaneous retainer affixed to said length closer to said second end than said first end;
said first subcutaneous retainer adapted to more easily penetrate a patient's skin in a first direction in which said first end extends, than in a second direction opposite said first direction; and
said second subcutaneous retainer adapted to more easily penetrate a patient's skin in an advancing direction in which said second end extends, than in a retreating direction opposite said advancing direction.

24. The device of claim 23 wherein said elongate length of material is flexible.

25. The device of claim 24 wherein said elongate length of material is elastic.

26. The device of claim 23 wherein said elongate length of material includes a substantially rigid cross bar extending between a pair of substantially rigid posts, with said subcutaneous retainers in the form of barbs on said posts.

27. The device of claim 23 wherein each said subcutaneous retainer is formed of a bio-absorbable material.
